(19) United States
(12) Patent Application Publication
(10) Pub. No.: US 2020/0324025 A1
(43) Pub. Date: Oct. 15, 2020

(54) COMPOSITIONS CONTAINING BONE MORPHOGENIC PROTEINS AND METHODS THEROF

(71) Applicant: GLOBUS MEDICAL, INC., AUDUBON, PA (US)

(72) Inventors: Joshua Mealy, King of Prussia, PA (US); Michael Oddo, Malvern, PA (US); Archana Bhat, Phoenixville, PA (US)

(21) Appl. No.: 16/839,305

(22) Filed: Apr. 3, 2020

Related U.S. Application Data
(60) Provisional application No. 62/831,949, filed on Apr. 10, 2019.

(51) Int. Cl. A61L 27/38; A61L 27/10; A61L 27/20; A61L 27/22; A61L 27/36; A61L 27/52; A61L 27/54

(52) U.S. Cl. CPC A61L 27/3821; A61L 27/10; A61L 27/20; A61L 27/22; A61L 27/3637; A61L 2420/02; A61L 27/52; A61L 27/54; A61L 2430/38; A61L 2430/02; A61L 27/3847

(57) ABSTRACT

Biomaterials, implants made therefrom, methods of making the biomaterial and implants, methods of promoting cartilage, tissue, bone or wound healing in a mammal by administering the biomaterial or implant to the mammal, and kits that include such biomaterials, implants, or components thereof. For example, the composition may include or be combined with bone morphogenic proteins.

### CROSS REFERENCE TO RELATED APPLICATIONS

This application is a non-provisional application which claims priority to provisional application Ser. No. 62/831,949 filed on Apr. 10, 2019, which is incorporated in its entirety herein.

### TECHNICAL FIELD

The present disclosure relates generally to bone and tissue healing biomaterials, and in particular, compositions and kits containing bone morphogenic proteins (BMPs). The disclosure also relates to methods of making the materials and implants, and methods of promoting bone, cartilage, or wound healing in a mammal by administering the biomaterial or implant to the mammal.

### BACKGROUND

Bone, cartilage, or tissue grafting is a surgical procedure that replaces missing bone, cartilage, or tissue and/or repairs bone, cartilage, or tissue. Bone, cartilage, and tissue generally have the ability to regenerate well but may require a scaffold or other growth enhancers to do so effectively. Grafts may be allograft (e.g., cadaveric origin or live donors), autologous (e.g., bone or tissue harvested from the patient's own body), or synthetic. Bone, cartilage, and/or tissue grafts may be resorbed and replaced as the natural bone, cartilage, or tissue heals over time.

Successful biomaterials may include osteoconduction (guiding the reparative growth of the natural bone), osteoinduction (encouraging undifferentiated cells to become active osteoblasts), and/or osteogenesis (living bone cells in the graft material contributing to bone remodeling). Although traditional grafts may exhibit certain advantages, traditional allograft may not exhibit the properties desired, may be difficult to obtain, or may not be in a shape or form suitable for implantation.

### SUMMARY

To meet this and other needs, BMP-containing biomaterials described herein may be configured to promote tissue, bone, and/or cartilage healing and repair. The compositions or implants prepared therefrom may include one or more bone morphogenic proteins (BMPs). The biomaterial compositions may be particularly suitable for use in bone or other tissue healing, for example, during a surgical procedure.

Biological growth factors may be utilized to accelerate and increase bone formation in spinal fusions, for example. Due to their significant osteoinductive properties, bone morphogenic proteins may be effective in conjunction with carrier systems for spinal fusion. As a result of rapid initial release and supraphysiologic doses in other systems, previous carriers have led to significant adverse events. These issues range from dysphagia and local swelling all the way to development of malignant tumors. To address these challenges, there is a need for a controlled release carrier system to lower the overall dose and provide a controlled release of BMPs. A composition, kit, or device that ensures a slow, controlled release of smaller doses of BMP while maintaining BMP bioactivity could address the stigmas associated with BMP therapies, and provide improvement on previous BMP/carrier systems.

According to one embodiment, a kit for aiding tissue regeneration includes a bone morphogenic protein solution; and a dry carrier mixture. The dry carrier mixture may include carboxymethylcellulose, bioactive glass or calcium-based granule, and/or optionally alginate. In particular, the carrier mixture may include 25-50% carboxymethylcellulose, 50-75% bioactive glass or calcium-based granule, and 0-25% alginate. The bone morphogenic protein solution may have a concentration ranging from about 10-10000 ug/mL, for example.

According to another embodiment, a method of preparing an implantable composition for aiding tissue regeneration may include combining a dry carrier mixture, for example, containing 25-50% carboxymethylcellulose and 50-75% bioactive glass or calcium-based granule with a bone morphogenic protein solution to form the implantable composition. The implantable composition may be in the form of a putty. Before combining the bone morphogenic protein solution with the dry carrier mixture, autologous bone marrow aspirate may be obtaining from a patient and combined with the bone morphogenic protein solution. The autologous bone marrow aspirate may be combined with a calcium chloride solution or thrombin solution prior to combination with the bone morphogenic protein solution in order to coagulate the bone marrow aspirate. According to another embodiment, alginate may be combined with the bone morphogenic protein solution before combining the bone morphogenic protein solution with the carrier mixture. The alginate may be combined with a calcium chloride solution prior to combining the alginate with the bone morphogenic protein solution in order to gel the alginate.

According to yet another embodiment, a method of promoting bone, tissue, or wound healing in a mammal may include providing a composition, for example, including one or more BMPs; and administering the composition into a target repair site to facilitate repair or regeneration of tissue at the target repair site. The target repair site may be an injury or defect in the spine and the tissue being regenerated may be bone or intervertebral disc.

According to yet another embodiment, a kit includes one or more of the components, compositions, or implants described herein, retrieval kits, trays, syringes, or other components for combining and administering the biomaterial components. In addition, the kit may include other components known in the art, including, but not limited to, additional carriers or scaffolds, cages (e.g., titanium and/or polyether ether ketone (PEEK) spacers), allograft spacers, cell culture media, phosphate buffered saline (PBS), a tissue culture substrate, retrieval tools, harvesting tools, implantation tools, or the like.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 depicts a process for a kit including a carrier mixture and a BMP solution, when mixed together form a putty mixture according to one embodiment;

FIG. 2 depicts a process for preparing a stabilized putty containing BMP according to another embodiment; and

FIG. 3 depicts a process for preparing a stabilized putty containing BMP according to yet another embodiment.

### DETAILED DESCRIPTION

The present disclosure relates generally to BMP-containing biomaterial compositions and implants made therefrom that may be used in a variety of surgical procedures. The disclosure also relates to methods of making the compositions and implants, and methods of promoting bone, tissue, or wound healing in a mammal by administering the biomaterial or implant to the mammal. The disclosure further relates to kits that include one or more of the biomaterials, implants, retrieval kits, tools and trays for mixing and combining ingredients, and other components thereof.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments of modifications thereof are contemplated as falling within the scope of this disclosure and equivalents thereto.

In describing example embodiments, specific terminology is employed for the sake of clarity. However, the embodiments are not intended to be limited to this specific terminology. Unless otherwise noted, technical terms are used according to conventional usage.

As used herein, “a” or “an” may mean one or more. As used herein “another” may mean at least a second or more. As used herein, unless otherwise required by context, singular terms include pluralities and plural terms include the singular.

As used herein and in the claims, the terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.”

Unless specified otherwise, all values provided herein include up to and including the endpoints given, and the values of the constituents or components of the compositions are expressed in weight percent or % by weight of each ingredient in the composition.

Each compound or name used herein may be discussed interchangeably with respect to its chemical formula, chemical name, abbreviation, acronym, etc. For example, BMP may be used interchangeably with bone morphogenic protein.

Embodiments described herein may be generally directed to BMP-containing biomaterial compositions, implants made therefrom, methods of making the same, and methods of using the same to promote healing of tissue, cartilage repair, and/or fusion of bone. Although compositions, biomaterials or implants may be discussed separately, it will be appreciated by one of ordinary skill in the art that the compositions or biomaterials described may be used in and of itself or may be used to create implants of different shapes, sizes, and orientations for a number of different clinical outcomes. Thus, the discussion of biomaterials or compositions may apply equally to the discussion on implants and vice versa.

According to one embodiment, the compositions or implants prepared therefrom may include bone morphogenic proteins (BMPs). The recombinant protein may be a bone morphogenetic protein, such as BMP-2, BMP-4, BMP-6, BMP-7, heterodimers thereof, and combinations thereof. In order to provide a controlled release of these potent proteins from a suitable carrier for surgical manipulation, the composition may utilize, for example, a putty to provide appropriate handling characteristics optionally combined with a synthetic or natural network to control long-term stability and BMP release.

When used for bone healing, the biomaterial compositions may be osteogenic, osteoinductive, osteoconductive, and/or osteostimulative, which may be advantageous for tissue or bone healing and repair. The biomaterials may be osteoconductive when the material serves as a scaffold that provides surface area for new bone or tissue growth. The biomaterials may be osteoinductive if they stimulate osteoprogenitor cells or induce mesenchymal stem cells to differentiate into osteoblasts that then begin new bone or tissue formation. Biomaterials may be osteogenic if they contain cells (e.g., viable cells) that are capable of bone regeneration. The biomaterial may be osteostimulative if the material accelerates the bone or tissue formation process.

When used for other tissue healing or regeneration, the biomaterial compositions may be configured to otherwise promote tissue healing. Tissue repair may be characterized by increased cell proliferation, capillary budding, and the synthesis of extracellular matrix (ECM) to fill in the damaged tissue. Thus, the biomaterial compositions may contain bioactive agents, precursors, or other properties suitable for promoting tissue healing and repair.

The composition may also be “biocompatible” as that term refers to the ability (e.g., of a composition or material) to perform with an appropriate host response in a specific application, or at least to perform without having a toxic or otherwise deleterious effect on a biological system of the host, locally or systemically. The biomaterial and/or implant or a portion thereof may be “biologically degradable” in that the material may be degraded by cellular absorption and/or hydrolytic degradation in a patient's body.

According to one embodiment, the biomaterial compositions may be configured to facilitate repair or regeneration of tissue, for example, bone, cartilage, or other tissue. In particular, the biomaterial compositions may facilitate repair or regeneration of tissue at a target repair site. The target repair site can be, for example, a void, gap, or other defect, or a surgeon created opening in bone, cartilage, between bones, or other structure or tissue location in a body of a patient. The biomaterial compositions may be configured to facilitate bone or other tissue growth at a target repair site. The biomaterial compositions may be configured to be directly implanted or otherwise disposed at and in contact with the target repair site. The patient and target repair site may be in a human, mammal, or other organism.

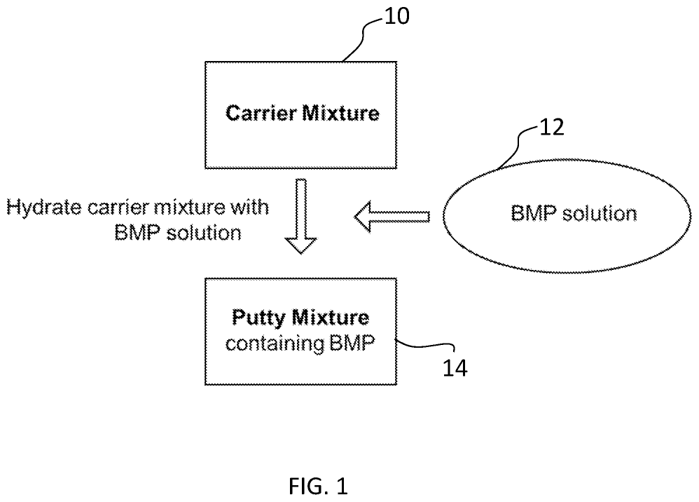

Turning now to FIG. 1, a composition for aiding tissue regeneration may be formed from a kit that includes a carrier mixture 10 and a BMP solution 12. Once the carrier mixture 10 is hydrated with the BMP solution 12, a biomaterial composition containing BMP 14 is obtained. The BMP-containing biomaterial composition 14 may be in the form of a putty, for example. Although a putty form is exemplified herein, it will be appreciated that the biomaterial composition may be in the form of a liquid, powder, gel, strip, extrudable, or other suitable version of the composition.

The kit includes a dry carrier mixture 10. In an exemplary embodiment, the carrier mixture 10 may include carboxymethylcellulose (CMC), a ceramic, such as bioactive glass or calcium-based granule, and/or optionally an alginate. The carrier mixture may comprise about 5-75% carboxymethylcellulose, about 10-60 carboxymethylcellulose, or about 25-50% carboxymethylcellulose, for example. The carrier mixture may comprise about 30-90%, about 40-80%, or about 50-75% bioactive glass and/or calcium-based granule, for example. The carrier mixture may optionally comprise alginate, for example, present at about 0-25%, about 1-15%, about 0.5-10%, or about 10-25% alginate.

The carrier mixture may comprise a ceramic, such as bioactive glass, a calcium-based granule, or a mixture thereof. For example, the ceramic may include ceramic mineral or inorganic filler useful for promoting bone formation. The ceramic component may include, but is not limited to, synthetic and naturally occurring inorganic fillers such as alpha-tricalcium phosphate, beta-tricalcium phosphate, tetra-tricalcium phosphate, dicalcium phosphate, calcium carbonate, barium carbonate, calcium sulfate, barium sulfate, hydroxyapatite (HA), biphasic calcium phosphate (e.g., composite between HA and 3-TCP), bioactive glass, and combinations and mixtures thereof. Tricalcium phosphate and bioactive glass share similar surface properties and show enhanced osteoconductivity in in vivo settings. Tricalcium phosphate has a similar composition to hydroxyapatite, but resorbs faster due to a lower calcium to phosphate (Ca/P) ratio. For example, hydroxyapatite has a Ca/P ratio of about 1.67 whereas tricalcium phosphate has a Ca/P ratio of about 1.5.

The ceramic component may provide osteoconductive and osteostimulative components to the construct by providing cell attachment sites and stimulating osteoblast proliferation and differentiation. The porosity, pore size, and pore geometry of the ceramic component can be tailored for the specific tissue regeneration application. In the case of bone tissue regeneration, a pore size of 100-500 μm may be targeted with an overall porosity of 60-80%.

If present, one or more ceramics may be included in the composition depending on the type or types of ceramic present, for example, in amounts ranging from about 30-90%, about 40-80%, or about 50-75% w/w.

The ceramic may comprise a bioactive glass. The bioactive glass may be configured to facilitate the regrowth of bone at the target repair site. In some embodiments, the bioactive glass can be an osteoconductive agent. Bioactive glass possesses osteostimulative properties, which may be useful in the regeneration of hard tissues. The bioactive glass can be disposed on, embedded within, and or mixed within the biomaterial composition. The bioactive glass can be any alkali-containing ceramic, glass, glass-ceramic, or crystalline material that facilitates bone formation after contact with a biological environment. Suitable bioactive glasses include sol gel derived bioactive glass, melt derived bioactive glass, silica based bioactive glass, silica free bioactive glass such as borate based bioactive glass and phosphate based bioactive glass, crystallized bioactive glass (either partially or wholly), and bioactive glass containing trace elements or metals such as copper, zinc, strontium, magnesium, zinc, fluoride, mineralogical calcium sources, and the like.

Exemplary bioactive glass can include bioglass 45S5 (46.1 mol % SiO₂, 26.9 mol % CaO, 24.4 mol % Na₂O and 2.5 mol % P₂O₅), 58S (60 mol % SiO₂, 36 mol % CaO and 4 mol % P₂O₅), 70S30C (70 mol % SiO₂, 30 mol % CaO), or a combination of the foregoing bioglass. The bioactive glass may take the form of fibers, granules, particles, or a combination thereof. The bioactive glass may be irregular in shape, for example. The bioactive glass may have a unimodal or bimodal particle size distribution. The bioactive glass may have a particle size, for example, ranging from about 1 to 1000 μm, about 50 to 750 μm, or about 75 to 500 μm. Particle size and distribution may be determined by routine techniques known in the art including sieve analysis or BET (Brunauer, Emmett and Teller) testing, for example. The bioactive glass particles may have a pore size of about 100 to 500 μm with an overall porosity of about 60-80%.

In certain embodiments, the ceramic comprises a calcium phosphate, such as beta-tricalcium phosphate (TCP). The calcium phosphate may be configured to facilitate regrowth of bone at the target repair site. In some embodiments, the calcium phosphate of the bone graft composition is an osteoinductive agent. The calcium phosphate can be in any suitable form. For example, the calcium phosphate can be in particulate or granular form. The calcium phosphate may have a particle size ranging from about 1 to 500 μm, about 25 to about 450 μm, about 50 to about 400 μm, about 75 to about 300 μm, or about 100 to about 250 μm, for example. The calcium phosphate may be porous or non-porous.

It is also envisioned one or more additional carriers, scaffold materials, or processing additives may be used with the composition. Suitable carriers, scaffolds, or additives may include, but are not limited to, demineralized bone matrix (DBM) or other bone-derived components, collagen including soluble and insoluble collagen, phospholipids, hyaluronic acid (HA), aggrecan, chondroitin sulfate, glycerin, glycerol, polyethylene glycol (PEG), hydrogels, poloxamers, polylactic acid (PLA), polylactic-co-glycolic acid (PLGA), other copolymers of the same family, and combinations thereof.

The carrier mixture 10 may be obtained using any suitable procedures and techniques known in the art. For example, components of the composition described herein may be mixed together to form the resulting, homogenous composition. The components may be combined, for example, at room temperature (e.g., about 20 and 26° C.) or other suitable conditions.

The kit includes a bone morphogenic protein solution. In particular, the carrier mixture may be combined with an exogenous recombinant protein component. The recombinant protein component may comprise a bone morphogenetic protein, such as BMP-2, BMP-4, BMP-6, BMP-7, heterodimers thereof, and combinations thereof. The bone morphogenic protein solution may be concentrated and/or purified in a sterile solution at an amount of about 1-10000 ug/mL, about 10-10000 ug/mL, about 100-1000 ug/mL, or about 1 mg/mL, for example.

The BMP solution and/or carrier may also contain growth factors or other biological agents, such as transforming growth factor (TGF-B), growth differentiation factor (GDF), platelet derived growth factor (PDGF), vascular endothelial growth factor (VEGF), insulin derived growth factor (IDGF), a keratinocyte derived growth factor (KDGF), or a fibroblast derived growth factor (FDGF), stem cells, and platelet rich plasma (PRP), to name a few. If desired, one or more active pharmaceutical ingredients or medicaments may be incorporated into the biomaterial or implant as well. Biological agents may be added in any suitable pharmaceutically acceptable and effective amounts known in the art.

In one embodiment, the bone morphogenic protein solution is combined with bone marrow aspirate (BMA). The bone marrow aspirate may be autologous bone marrow aspirate derived from the patient being treated. The autologous bone marrow aspirate may be combined with the bone morphogenic protein solution before being combined with the carrier mixture, for example. The autologous bone marrow aspirate may be coagulated prior to combination with the bone morphogenic protein solution. Coagulation of the BMA may be induced ex vivo through incubation, for example, at 25-37° C., optionally, through addition of exogenous clotting factors. For example, the autologous bone marrow aspirate may be coagulated with a Ca²⁺ (e.g., calcium chloride) or thrombin solution. Alternatively, the BMA may be coagulated in situ during or after surgical implantation.

The BMP solution (e.g., combined with coagulated BMA) may be combined with the carrier mixture before or during the time of surgery. The dry carrier mixture and the bone marrow aspirate/bone morphogenic protein solution may be combined at a ratio of about 3:1 to 1:3, or about 1:1 (dry carrier (g):BMA/BMP (mL)).

In another embodiment, the bone morphogenic protein solution is combined with an aqueous alginate. The alginate may be gelled prior to combination with the bone morphogenic protein solution. Gelation of the alginate may be accomplished with a calcium chloride solution, for example, or other suitable gelation agent. The gelled or aqueous alginate may be combined with the bone morphogenic protein solution before being combined with the carrier mixture. In one embodiment, 0.5-10% w/v alginate may be combined with the BMP solution.

The BMP solution (e.g., combined with gelled alginate) may be combined with the carrier mixture before or during the time of surgery. The dry carrier mixture and the alginate/bone morphogenic protein solution or gel may be combined at a ratio of about 3:1 to 1:3, or about 1:1 (dry carrier (g):alginate/BMP (mL)).

According to one embodiment, a method of preparing an implantable composition for aiding tissue regeneration includes combining a dry carrier mixture 10 with the bone morphogenic protein solution 12 to form the implantable composition 14. In particular, the dry carrier mixture 10 may be hydrated with the BMP solution 12 to form a putty mixture 14, which contains BMP. The putty mixture 14 may provide for a controlled release of the BMP in situ. A slow or controlled release of the BMP, while maintaining BMP bioactivity may provide for a more effective release of BMP while minimizing or avoiding adverse events.

The biomaterials described herein and/or implants formed therefrom are intended to be applied at a tissue, bone or cartilage repair sites, e.g., one resulting from injury or defect. The implant can be utilized in a wide variety of orthopedic, periodontal, neurosurgical, oral and maxillofacial surgical procedures. In particular, the biomaterials may be suitable for repairs of the vertebral column including spinal fusion and internal fixation; tumor surgery, e.g., deficit filling; discectomy; laminectomy; scoliosis, lordosis and kyphosis treatments. Possible clinical applications may include e.g., the treatment of spinal disc degeneration or disease, traumatic, pathologic, or stress fractures, congenital defects or fractures, or operative defects in any bone or between bones of the body.

The compositions and implants may be configured for use at various target repair sites within a body of a patient to facilitate bone, cartilage, and/or tissue growth therein. In some embodiments, the composition is configured for use at a target repair site in the patient's spine. For example, the composition can facilitate growth of bone between the body of a first vertebra and the body of a second vertebra to achieve interbody fusion of the two vertebrae. In a spinal fusion procedure, the composition may be used in conjunction with one or more mechanical supports (e.g., a cage or frame, spacer, plate, a plurality of screws and/or rods, or the like). Although the spine is described, the composition can be configured to be implanted into or at a target repair site in or at a different bone, tissue or other structures of the patient's body.

The term “treating” and the phrases “treatment of a disease” and “treatment of a condition” refer to executing a protocol that may include the use of the compositions, devices and methods herein and/or administering one or more biomaterials to a patient (human, normal or otherwise, or other mammal), in an effort to alleviate signs or symptoms of the disease or condition. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms and does not require a cure to the ailment.

The following examples are provided to further illustrate various non-limiting embodiments and techniques. It should be understood, however, that these examples are meant to be illustrative and do not limit the scope of the claims. As would be apparent to skilled artisans, many variations and modifications are intended to be encompassed within the spirit and scope of the invention.

### EXPERIMENTAL EXAMPLES

The examples provided below may allow for low to moderate doses of BMP for controlled release, for example, in a shapeable putty formulation

### Example 1: BMP Combined with BMA Carrier

Autologous bone marrow aspirate (BMA) collected during surgery is an attractive option for carrying BMP. BMA is non-immunogenic as it comes from an autologous source, and may be used to generate a coagulate network, which may provide encapsulation and affinity for a variety of growth factors, including BMP.

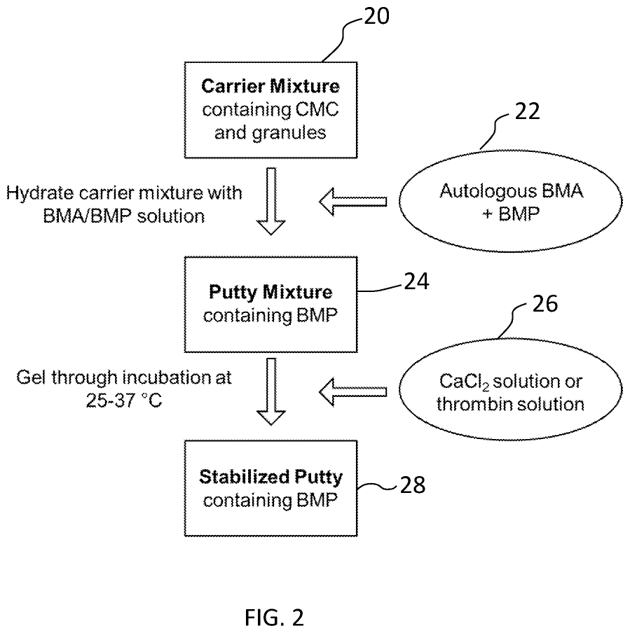

As shown in FIG. 2, a suitable method for making a synthetic BMP-containing composition is provided. Autologous BMA will be collected at the time or surgery, and combined directly with BMP, for example, at a concentration of 10-10000 μg/mL, as shown in step 22. This BMA/BMP solution 22 will hydrate a dry carrier mixture 20 containing carboxymethylcellulose (25-50% dry weight), bioactive glass or other calcium-based granule (50-75% dry weight), and alginate (0-25% dry weight) in a range from 3:1 to 1:3 (dry carrier (g):BMA (mL)) to yield a shapeable, cohesive putty mixture 24. As shown in step 26, coagulation of BMA may be induced ex vivo through incubation at 25-37° C., optionally, through addition of exogenous clotting factors such as thrombin or Ca²⁺ (e.g., CaCl₂)), in situ after surgical implantation, or any combination thereof. This results in a stabilized putty 28 containing BMP.

Product will be shipped as a 1 cc, 5 cc, and 10 cc kit containing dry mixture and a sterile BMP solution, stored at −20° C. to −80° C., for example.

### Example 2: BMP Combined with a Synthetic Matrix

Synthetic hydrophilic matrices may be used to entrap and retain BMPs, offering the advantage of tunable degradation and release properties through control of polymer properties, while maintaining biocompatible processing conditions that minimally affect protein activity.

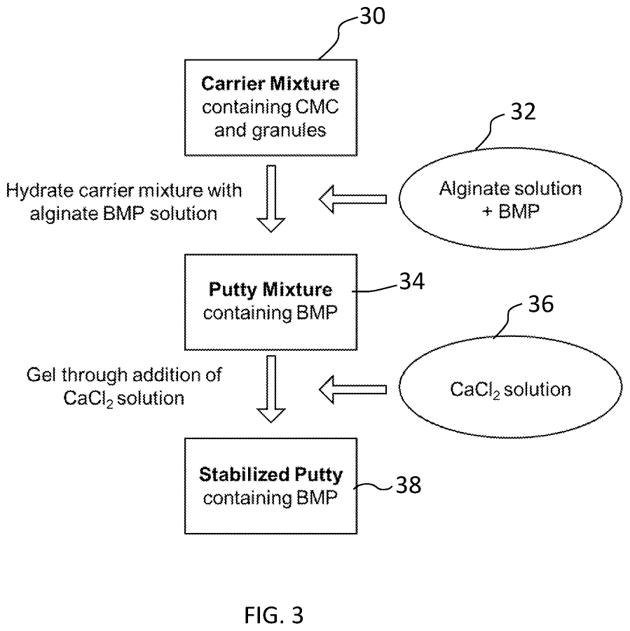

As shown in FIG. 3, a suitable method for making a synthetic BMP-containing composition is provided. An aqueous alginate solution (e.g., 0.5-10% w/v) will be combined with BMP (e.g., 10-10000 μg/mL) in step 32. This solution 32 will be added to a dry carrier mixture 30 containing carboxymethylcellulose (25-50% dry weight), bioactive glass or other calcium-based granule (50-75% dry weight), and optionally alginate (0-25% dry weight), in a range from 3:1 to 1:3 (dry carrier (g):alginate solution (mL)), to yield a shapeable, cohesive putty 34. Alginate gelation may be induced ex vivo via incorporation of CaCl₂) in the dry carrier mixture or through the addition of a CaCl₂) solution to stabilize putty and entrap BMP for sustained release (shown in step 36). This results in a stabilized putty 38 containing BMP.

Product will be shipped as a 1 cc, 5 cc, and 10 cc kit containing dry carrier, sterile alginate solution, and a sterile BMP solution stored at −20 to −80° C., for example.

Although the invention has been described in example embodiments, those skilled in the art will appreciate that various modifications may be made without departing from the spirit and scope of the invention. It is therefore to be understood that the inventions herein may be practiced other than as specifically described. Thus, the present embodiments should be considered in all respects as illustrative and not restrictive. Accordingly, it is intended that such changes and modifications fall within the scope of the present invention as defined by the claims appended hereto.

What is claimed is:

1. A kit for aiding tissue regeneration, the kit comprising:
a bone morphogenic protein solution; and
a dry carrier mixture, wherein the carrier mixture comprises 25-50% carboxymethylcellulose, 50-75% bioactive glass or calcium-based granule, and 0-25% alginate.

2. The kit of claim 1, wherein the bone morphogenic protein solution is present at 10-10000 ug/mL.

3. The kit of claim 1, further comprising autologous bone marrow aspirate, wherein the autologous bone marrow aspirate is configured to be combined with the bone morphogenic protein solution before being combined with the carrier mixture.

4. The kit of claim 3, further comprising a calcium chloride solution or thrombin solution, wherein the autologous bone marrow aspirate is configured to be coagulated with the calcium chloride solution or thrombin solution prior to combination with the bone morphogenic protein solution.

5. The kit of claim 3, wherein a ratio of the dry carrier mixture to the bone morphogenic protein solution combined with the bone marrow aspirate is configured to be combined as 3:1 to 1:3 (dry carrier (g):BMA (mL)).

6. The kit of claim 1, further comprising 0.5-10% w/v alginate, wherein the alginate is configured to be combined with the bone morphogenic protein solution before being combine with the carrier mixture.

7. The kit of claim 6, further comprising a calcium chloride solution, wherein the alginate is configured to be gelled with the calcium chloride solution prior to combination with the bone morphogenic protein solution.

8. The kit of claim 6, wherein a ratio of the dry carrier mixture to the bone morphogenic protein solution combined with the alginate is configured to be combined as 3:1 to 1:3 (dry carrier (g):alginate (mL)).

9. A method of promoting bone or tissue in a mammal, the method comprising:
mixing the bone morphogenic protein solution and the dry carrier mixture from the kit of claim 1 to form a putty; and
contemporaneously, administering the putty into a target repair site to facilitate repair or regeneration of tissue at the target repair site.

10. A method of preparing an implantable composition for aiding tissue regeneration, the method comprising:
combining a dry carrier mixture containing 25-50% carboxymethylcellulose and 50-75% bioactive glass or calcium-based granule with a bone morphogenic protein solution to form the implantable composition.

11. The method of claim 10, wherein the implantable composition is a putty.

12. The method of claim 10, wherein the bone morphogenic protein solution is present at 10-10000 ug/mL.

13. The method of claim 10, wherein before combining the bone morphogenic protein solution with the dry carrier mixture, obtaining autologous bone marrow aspirate from a patient and combining the bone morphogenic protein solution with the bone marrow aspirate.

14. The method of claim 13, further comprising combining the autologous bone marrow aspirate with a calcium chloride solution or thrombin solution prior to combination with the bone morphogenic protein solution in order to coagulate the bone marrow aspirate.

15. The method of claim 13, wherein a ratio of the dry carrier mixture to the bone morphogenic protein solution combined with the bone marrow aspirate is 3:1 to 1:3 (dry carrier (g):BMA (mL)).

16. The method of claim 10, further comprising combining 0.5-10% w/v alginate with the bone morphogenic protein solution before combining the bone morphogenic protein solution with the carrier mixture.

17. The method of claim 16, further comprising gelling the alginate with a calcium chloride solution prior to combining the alginate with the bone morphogenic protein solution.

18. The method of claim 16, wherein a ratio of the dry carrier mixture to the bone morphogenic protein solution combined with the alginate is 3:1 to 1:3 (dry carrier (g):alginate (mL)).

19. A method of promoting bone or tissue in a mammal, the method comprising:
providing the implantable composition obtained from the method of claim 10; and
administering the composition into a target repair site to facilitate repair or regeneration of tissue at the target repair site.

20. The method of claim 19, wherein the target repair site is an injury or defect in the spine and the tissue being regenerated is bone.
